(19) United States
(12) Patent Application Publication
(10) Pub. No.: US 2005/0089948 A1
(43) Pub. Date: Apr. 28, 2005

(54) Decontamination and concentration kit for mycobacteria

(76) Inventor: Tanil Zuhtu Kocagoz, Istanbul (TR)

Correspondence Address: MEYERTONS, HOOD, KIVLIN, KOWERT & GOETZEL, P.C., P.O. BOX 398, AUSTIN, TX, 78767-0398, US

(21) Appl. No.: 10/947,915

(22) Filed: Sep. 23, 2004

(30) Foreign Application Priority Data
Oct. 23, 2003 (TR) TR 2003 01829

(51) Int. Cl. G01N 33/554; G01N 33/569; C12Q 1/04; C12N 1/20

(52) U.S. Cl. 435/34; 435/253.1

(57) ABSTRACT

A kit is provided for use in mycobacterial testing that provides pre-packaged containers of reagent used in mycobacterial decontamination and concentration procedures. A method of using the kit is also provided. Using the kit embodied herein removes the need to access stock reagents in the laboratory and may substantially reduce the likelihood of sample cross-contamination.

### BACKGROUND

1. Field of Invention

The embodiments presented herein generally relate to mycobacterial testing. More particularly, the embodiments presented herein relate to a kit and method for decontaminating samples of non-mycobacterial contaminants and concentrating mycobacteria present in the samples.

2. Description of Related Art

Mycobacterial infection continues to be a major health problem around the world. It is estimated that every year ten million people become tuberculosis patients and three million die of this disease. Definitive diagnosis of mycobacterial infection typically requires isolation and identification of mycobacterial species from patient-derived clinical samples that may cause similar diseases. Obtaining pathogenic mycobacteria from biological specimens as pure culture may also be an important step toward obtaining a definitive diagnosis of mycobacterial infection, in determining and planning appropriate therapeutic intervention, or in determining the susceptibility of a clinical isolate to antimycobacterial drugs.

Clinical samples, such as sputum or other body fluids obtained from patients, are often contaminated in situ or during the specimen collection and/or transport process with diverse microbial flora that may make it difficult to subsequently isolate, culture and identify mycobacteria. Therefore, in certain instances, it may be advantageous to first decontaminate the samples in order to remove contaminating microbial flora. A commonly used procedure to decontaminate microbial flora from samples and concentrate mycobacterium is an adaptation of the N-acetyl-L-cysteine-sodium hydroxide (NALC-NaOH) decontamination and concentration procedure as outlined in Kubica et al, 1963, Mosby, 1994 and Heifets and Good, 1994, all of which are incorporated by reference as though fully set forth herein.

Processing the samples by the NALC-NaOH procedure decontaminates the samples by killing non-mycobacterial flora that are susceptible to sodium hydroxide while mycobacteria, which are typically insensitive to conditions of high alkalinity, survive. Advantageously, the inclusion of a reducing agent such as N-acetyl-cysteine helps to liquefy viscous clinical samples, which may facilitate the sedimentation of bacilli during subsequent centrifugation or other concentration steps.

Following decontamination and concentration, the recovered material may be subjected to further testing to detect mycobacteria. For example, the recovered material may be cultured according to the methods described in U.S. patent application Ser. No. 10/683,565, which is incorporated by reference as though fully set forth herein, which describes a rapid test to detect mycobacteria using colorimetric method. The recovered material may also be further cultured using conventional culturing methods familiar to practitioners of the art, and subjected to microscopy or molecular analysis including biochemical analysis or sensitive molecular identification techniques such as polymerase chain reaction (PCR) or other nucleic acid sequence based analyses (NASBA).

Typically, microbiology laboratories performing tests on or for mycobacteria use reagents that are prepared by the laboratories, or are purchased commercially, in bulk. The reagents and buffers used during this procedure are stored in large containers and may typically be accessed numerous times daily by several laboratory personnel. Such bulk reagents and solutions, while convenient, are a prime source of sample cross-contamination, since they themselves may frequently become contaminated after repeated use. Sample cross-contamination may be particular problematic in laboratories where sensitive genetic procedures, such as PCR, are routinely employed. Adequately guarding against sample cross-contamination thus requires extensive quality control measures, frequent replacement of bulk solutions and decontamination of storage containers and laboratory equipment, resulting in an unnecessary expenditure of time, energy and resources.

### SUMMARY

Described herein is a kit that enables the rapid decontamination and concentration of mycobacteria from biological samples using sets of single use pre-measured reagents.

In one embodiment, a kit for decontaminating and concentrating mycobacteria may include one or more sets of single use containers that contain measured amounts of reagents required for the decontaminating and concentrating procedure. In an embodiment, each set of single use containers may be used to decontaminate and concentrate only one biological sample, after which the set of single use containers is discarded. Such embodiments may substantially decrease the likelihood that a biological sample may become cross-contaminated with mycobacteria from a separate biological sample.

In an embodiment, a set of single use containers may include three containers. In an embodiment, the three containers may include a first container containing a mucolytic compound, a second container containing a bactericidal compound and a third container containing a buffer. In an embodiment, the containers may be conical polypropylene tubes that may be suitable for use in standard laboratory centrifuges. In an embodiment, mucolytic compounds may include thiol-containing reducing compounds such as N-acetyl-cysteine. In an embodiment, bactericidal compounds may include alkaline compounds such as sodium hydroxide solutions. In an embodiment, a buffer may include a solution with a buffering capacity in the physiological pH range of approximately 6.5 to 7.5, such as a phosphate buffer solution.

In another embodiment, rapid decontamination and concentration of mycobacteria from biological samples may be achieved using a kit embodied herein. In an embodiment, a set of three single use containers may be provided to process one biological sample. In an embodiment, a biological sample is added to the first container containing a mucolytic compound such as a thiol-containing compound. A bactericidal compound from the second container may be added to the biological sample in the first container. The volume of the biological sample may be adjusted with a buffer solution added from the third container. The mycobacteria bacilli may be collected by centrifugation. A container containing a disinfectant solution may be included with the kit, and may be used to decontaminate the first container prior to discarding it.

### DETAILED DESCRIPTION

Proper, timely and reliable diagnosis of mycobacterial infections is of importance to public health. Typically, testing for mycobacteria occurs in diagnostic laboratories equipped to handle a large number of patient specimens.

Collecting high quality clinical samples from patients undergoing tests for the presence of mycobacteria forms the basis for diagnosis of mycobacterial infections and helps make an appropriate determination of therapeutic intervention. Central to the ability to make accurate and reliable diagnoses of mycobacterial infection is the ability to avoid cross-contamination of patient samples. Cross contamination of samples may easily occur in a testing facility where large numbers of patient samples are stored in relatively close proximity, and where testing reagents and equipment are handled by numerous laboratory personnel and may come into contact with the large numbers of patient samples. Particular care must be taken to avoid sample cross-contamination in laboratories where favored diagnostic methods include PCR, or where mycobacteria from different patient samples must be cultured for prolonged periods of time. As used herein, the term “sample cross-contamination” generally refers to the contamination of a patient sample with bacteria or other material from a separate sample. For the purposes illustrated herein, a separate sample may include a different sample derived from the same patient or a sample from a different patient.

Sample cross-contamination may result in patient samples falsely testing positive for the presence of mycobacteria and may necessitate prolonged and costly disruption to testing services while equipment is adequately decontaminated and testing material and reagents are replaced. Additionally, sample cross-contamination may necessitate collecting additional samples from patients, which may substantially add to the costs associated with retesting and, potentially, the need to have test results confirmed by independent testing laboratories. It is therefore desirable to devise a simple and economical system that substantially reduces the risk of sample cross-contamination, yet retain the ability to rapidly process large numbers of patient samples with quality-assured batches of stock reagents.

As embodied herein, testing for mycobacteria may include, but is not limited to, testing for mycobacteria of the M. tuberculosis complex, including M. tuberculosis, M. leprae, M. africanum, M. bovis or M. ulcerans, or mycobacteria of the Runyon Groups I-IV. Procedures used to identify mycobacterial species are well understood in the art and may include the use of culturing methods, microscopic analysis, biochemical analysis, molecular diagnostic methods including NASBA and PCR, or combinations thereof.

During a typical mycobacterial testing procedure, a specimen is collected from a patient in a sterile collection container. In the case of testing for M. tuberculosis, sputum is the specimen most often collected, although other suitable specimens may include, but are not limited to, bronchoalveolar lavage fluid (BAL), gastric lavage fluid (GAL), blood, pleural, pericardial, spinal or peritoneal fluids, urine or aspirates from patient lesions. In some instances, it may be desirable to test excised organ tissue, such as a tissue excised during a biopsy or necropsy procedure, for the presence of mycobacteria. Mycobacteria may be recovered from organ tissue by finely mincing the tissue and/or homogenizing the tissue in a Teflon-to-glass or a glass-to-glass tissue homogenizer according to routine procedure in the art.

Typically, biological samples suspected of containing mycobacteria may be collected in a clean sterile container according to practices widely known in the art. Since most patient samples are collected from non-sterile sites, they may frequently be contaminated with non-mycobacterial flora. As used herein, “non-mycobacterial flora” generally refers to naturally occurring non-mycobacterial microorganisms that are present in a biological sample and that may interfere with the identification or manipulation of mycobacteria from said biological sample. As used herein, non-mycobacterial flora may also be referred to as “microbial flora.” It may sometimes be necessary to kill or remove microbial flora prior to performing subsequent procedures. As used herein, killing or removing microbial flora in a sample containing mycobacteria may generally be referred to as “decontaminating” the sample. Typically, decontaminating a sample may also be performed in conjunction with concentrating mycobacteria in the sample. Concentrating mycobacteria in the sample may facilitate subsequent procedures such as culturing, or performing certain NASBA procedures, such as hybridization.

In some embodiments, it may be necessary to process biological samples to facilitate subsequent procedures. Processing the biological samples may include steps to substantially homogenize or liquefy the sample. Liquefying the sample may be required when handling viscous specimens such as, for example, sputum or other viscous bodily fluids. Liquefying, or reducing the viscosity of a sample may improve the recovery of mycobacteria from the sample, particularly when mycobacteria are collected by centrifugation. In an embodiment, reducing the viscosity of a sample may include using a mucolytic compound. As used herein, the term “mucolytic compound” generally refers to a compound that substantially breaks down, degrades or otherwise liquefies and reduces the viscosity of mucous. In an embodiment, a mucolytic compound may include a compound that substantially reduces inter- and intramolecular disulfide bonds in mucins and other protein components of mucous. In an embodiment, a mucolytic compound may include a reducing compound such as, for example, a thiol-containing compound. Non-limiting examples of thiol-containing compounds that may be suitable muclolytic compounds in the embodiments presented herein include cysteine, glutathione, N-acetyl-cysteine, N-acetyl-L-cysteine, N-isobutyrylcysteine, dithiotreithol (DTT) or combinations thereof. In an embodiment, a muclolytic compound may include N-acetyl-cysteine.

Additionally, some embodiments may require a decontamination step that kills or otherwise substantially removes microbial flora from the sample. After these steps, the remaining mycobacteria may be concentrated by a centrifugation step. In an embodiment, decontaminating biological samples may include treating the samples with a bactericidal compound. Mycobacteria may resist the action of certain bactericidal compounds. In an embodiment, such compounds may be used to selectively kill the microbial flora of a biological sample while allowing the mycobacteria to survive. Examples of such bactericidal compounds adequate for the embodiments presented herein may include, but are not limited to, zephiran-trisodium phosphate, alkaline compounds such as alkali metal hydroxides (e.g. sodium hydroxide), acidic compounds (e.g. oxalic or sulfuric acids), cetylpyridnium chloride compounds, or combinations thereof.

Mycobacterial decontamination and concentration procedures employed by most mycobacteria testing laboratories include modifications of the N-acetyl-L-cysteine sodium hydroxide (NALC-NaOH) procedure. A typical NALC-NaOH procedure may include mixing a biological sample with a volume of NALC-NaOH solution that is approximately equal to the volume of the biological sample and that contains about 0.1-1.0 N NaOH as a decontaminating compound and about 5-50 wt % N-acetyl-L-cysteine as a mucolytic compound. In an embodiment, adding mixing aides such as, for example, glass beads to the sample may substantially aide in liquefying and decontaminating the biological sample. In an embodiment, an NALC-NaOH solution may also contain about 0.01-1.0 M trisodium citrate. After the NALC-NaOH and biological sample are homogeneously mixed, the mixture may be left at room temperature for a period generally not exceeding about 15 minutes, after which time the liquefied and decontaminated sample may be diluted with water, or with an appropriate volume of a physiological buffer such as, for example a phosphate buffer, phosphate buffered saline, or any such buffer solution with a pH that is in the range of approximately 6.5 to 7.5. Diluting the NALC-NaOH and biological sample mixture with water or with a buffer solution may substantially reduce the alkalinity of the mixture and inhibit the bactericidal activity of the NALC-NaOH solution. In an embodiment, the NALC-NaOH procedure may be carried out in a container that is adapted for use in a standard laboratory centrifuge. In an embodiment, suitable containers for this purpose may include 50 mL conical polypropylene centrifuge tubes such as, for example, a conical polypropylene centrifuge tubes available commercially from Falcon® or other vendor of laboratory supplies, as is widely recognized in the art. The diluted biological sample may be centrifuged at a speed and for period of time that will substantially sediment the mycobacterial bacilli that are suspended in the sample. In an embodiment, centrifugation may be carried out at from about 1000-5000×G for about 5-30 minutes. To prevent aerosolization of the sample it may be desirable to perform the centrifugation step using airtight centrifuge buckets for and/or performing the centrifugation step in a refrigerated centrifuge. Following the centrifugation step, the supernatant may be discarded, and the sediment containing viable mycobacteria may be collected with an inoculation loop, or may be resuspended in water, buffer, or an appropriate growth medium.

It is an object of the embodiments presented herein to provide a kit for performing the NALC-NaOH decontamination and concentration procedure using sets of single use, prepackaged and pre-measured reagents. Each set of single use, prepackaged and pre-measured reagents may be used to decontaminate and concentrate only one biological sample. After the sample is processed, the single use set of reagents is discarded. In an embodiment, such sets of single use, prepackaged and pre-measured reagents may be provided in separate containers such as, for example, conical polypropylene centrifuge tubes. In these embodiments, the use of single use sets of prepackaged and pre-measured reagents may substantially reduce the risk of sample cross-contamination.

An illustrative example of an embodiment of a kit for performing the NALC-NaOH decontamination and concentration procedure may include the following:
A kit for performing decontamination and concentration procedures on 25 biological samples may include
25×50 mL polypropylene tubes containing a measured amount of N-Acetyl-L-cysteine and glass beads.
25×10 mL sodium hydroxide—trisodium citrate solution in plastic tubes.
25×50 mL sterile phosphate buffer, pH 6.8-7.0 in polypropylene tubes.

In an embodiment, one or more volumes of a disinfecting solution may be provided with the kit to disinfect any materials and/or surfaces that were used during the procedure. For example, in an embodiment, two 200 mL bottles of a sodium hypochlorite disinfectant solution may be included. The disinfectant may be used to wash the polypropylene tubes that held the biological sample and that were used to perform the decontamination and concentration procedure prior to discarding the tubes. In an embodiment, the materials included in the kit may be provided in one or more boxes to facilitate storage and handling.

In further embodiments, the kit may also be provided with positive and negative control samples. For example, some embodiments may include positive control samples that include respiratory secretions spiked with mycobacteria. Some embodiments may include negative controls that include respiratory secretions spiked with Escherichia coli and/or Staphylococcus aureus.

The following examples serve to illustrate embodiments of a kit for decontaminating and concentrating biological samples and a method of using same according to the foregoing embodiments. The examples are illustrative and should not be considered limiting.

### EXAMPLE 1

For Sputum and Samples Other Than Urine

A maximum sample volume of 10 mL is collected from a patient. Suitable patient samples include samples such as sputum, bronchoalveolar fluid, gastric lavage fluid, pleural, pericardial, or peritoneal fluids. The sample is transferred from the collection cup to a sterile 50 mL conical polypropylene first tube containing approximately 40 mg of N-acetyl-L-cysteine, and glass beads (approximately 4 mm in diameter).

A volume of a solution of 3 wt % sodium hydroxide and 1.47 wt % trisodium citrate·3H₂O that is approximately equal to the volume of the patient sample is taken from a second sterile polypropylene tube and added to the first tube containing the mixture of the patient sample, N-acetyl-L-cysteine and the glass beads. The first tube is capped the contents thereof are agitated either by shaking or by using a bench top vortex to homogenize the patient sample. The first tube is left standing at room temperature for 15 minutes

The contents of the first tube are adjusted to a volume of 50 mL with sterile phosphate buffer (0.676 (1/15) M Na₂HPO₄+KH₂PO₄, pH 6.8-7.0) from a third polypropylene tube.

The first tube is centrifuged at approximately 3500×G (which corresponds to 4000 rpm in a centrifuge with a rotor radius of 20 cm) with airtight centrifuge buckets for approximately 15 minutes. To help prevent aerosolization of the patient sample, the centrifugation step may be performed in a refrigerated centrifuge.

The supernatant of the first tube is decanted into a disinfectant solution. Approximately 5 mL of the sterile phosphate buffer from the third tube is added to the sediment and glass beads in the first tube and the tube is then vortexed to resuspend the sedimented material. This suspension may now be used for culture, microscopy or other molecular diagnostic methods.

Once the procedure is complete, the first tube may be disinfected before discarding by adding approximately 10 mL of a stock sodium hypochlorite solution provided with the kit. The first tube is capped and agitated vigorously to ensure that the entire inner surface of the first tube is contacted with the disinfectant solution.

The first, second and third tubes are then discarded. If additional patient samples are to be processed, a new set of first, second and third tubes are used.

### EXAMPLE 2

For Urine Samples

An early morning urine sample is collected from a patient. Up to 50 mL of urine is transferred from the collection cup into the first tube containing approximately 40 mg of N-acetyl-L-cysteine, and glass beads. The first tube is centrifuged at 3500×G in airtight centrifuge buckets for approximately 15 minutes. A refrigerated centrifuge may be used to minimize aerosolization of the patient sample.

The supernatant in the first tube is discarded according to established protocols for disposing of biohazardous body fluids. Approximately 3 mL a solution of 3 wt % sodium hydroxide and 1.47 wt % trisodium citrate·3H₂O from a second sterile polypropylene tube are added to the sediment and glass beads remaining in the first tube. The first tube is capped and vortexed to resuspend the sedimented material. The first tube is left standing at room temperature for 15 minutes.

The contents of the first tube are adjusted to a volume of 50 mL with sterile phosphate buffer (0.676 (1/15) M Na₂HPO₄+KH₂PO₄, pH 6.8-7.0) from a third polypropylene tube.

The first tube is centrifuged at approximately 3500×G (which corresponds to 4000 rpm in a centrifuge with a rotor radius of 20 cm) with airtight centrifuge buckets for approximately 15 minutes. To help prevent aerosolization of the patient sample, the centrifugation step may be performed in a refrigerated centrifuge.

The supernatant of the first tube is decanted into a disinfectant solution. Approximately 2.5 mL of the sterile phosphate buffer from the third tube is added to the sediment and glass beads in the first tube and the tube is then vortexed to resuspend the sedimented material. This suspension may now be used for culture, microscopy or other molecular diagnostic methods.

Once the procedure is complete, first tube may be disinfected before discarding by adding approximately 10 mL of a stock sodium hypochlorite solution provided with the kit. The first tube is capped and agitated vigorously to ensure that the entire inner surface of the first tube is contacted with the disinfectant solution.

The first, second and third tubes are then discarded. If additional patient samples are to be processed, a new set of first, second and third tubes are used.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes am be made in the elements described herein without departing from the spirit and scope of the invention as described in the following claims. In addition, it is to be understood that features described herein independently may, in certain embodiments, be combined.

### REFERENCES

1- Kubica G P, Dye W E, Cohn M L, Middlebrook G. Sputum digestion and decontamination with N-acetyl-L-cysteine-sodium hydroxide for culture of mycobacteria. 1963. Am. Rev. Respir. Dis. 87:775-779.

2- N-acetyl-L-cysteine-sodium hydroxide method for liquefaction and decontamination of specimens. Bailey & Scott's Diagnostic Microbiology, Ninth Edition. Mosby-Year Book Inc. St. Louis, Mo. USA. 1994, p: 600.

3- Heifets L B, Good R C. Current laboratory methods for the diagnosis of tuberculosis. In “Tuberculosis” Ed. Bloom B R. ASM Press, Washington D.C. 1994. 85-110.

1. A kit for decontaminating and concentrating mycobacteria in biological samples comprising one or more single use sets of containers, wherein each single use set of containers comprises:
a first container comprising a measured amount of a mucolytic compound,
a second container comprising a measured amount of a bactericidal compound, and
a third container comprising a measured amount of a buffer solution,
wherein each single use set of containers is used to decontaminate and concentrate mycobacteria in only one biological sample.

2. The kit of claim 1, further comprising a container comprising a disinfectant solution.

3. The kit of claim 2, wherein the disinfectant solution comprises a sodium hypochlorite solution.

4. The kit of claim 1, wherein the first container further comprises a mixing aid.

5. The kit of claim 4, wherein the mixing aid comprises glass beads.

6. The kit of claim 1, wherein the mucolytic compound comprises a thiol-containing compound.

7. The kit of claim 6, wherein the thiol-containing compound is selected from the group of thiol-containing compounds comprising cysteine, glutathione, N-acetyl-cysteine, N-isobutyrylcysteine.

8. The kit of claim 6, wherein the thiol-containing compound comprises N-acetyl-cysteine.

9. The kit of claim 1, wherein the bactericidal compound comprises a hydroxide containing solution.

10. The kit of claim 1, wherein the bactericidal compound comprises a sodium hydroxide solution.

11. The kit of claim 1, wherein the bactericidal compound comprises a sodium hydroxide and sodium citrate solution.

12. The kit of claim 1, wherein the buffer solution comprises a phosphate buffer solution.

13. The kit of claim 1, wherein the buffer solution comprises a phosphate buffer solution, wherein the pH of the buffer solution is from about 6.5 to about 7.5.

14. The kit of claim 1, wherein the biological sample comprises sputum, bronchoalveolar fluid, gastric lavage fluid, pleural, pericardial or peritoneal fluids or urine.

15. The kit of claim 1, wherein one or more of the containers comprise tubes adapted for use in a centrifuge.

16. A method for decontaminating and concentrating mycobacteria in a biological sample using a kit, the method comprising:
adding a biological sample to a first container comprising a measured amount of a mucolytic compound,
mixing the biological sample and the mucolytic compound,
adding and mixing a measured amount of a bactericidal compound from a second container to the first container,
adding and mixing a measured amount of a buffer solution from a third container to the first container,
wherein the first, second and third containers comprise a set of single use containers of the kit, wherein each single use set of containers is used to decontaminate and concentrate mycobacteria in only one biological sample.

17. The method of claim 16, wherein the first container further comprises glass beads.

18. The method of claim 16, wherein the mucolytic compound comprises a thiol-containing compound.

19. The method of claim 16, wherein the thiol-containing compound is selected from the group of thiol-containing compounds comprising cysteine, glutathione, N-acetyl-cysteine, N-isobutyrylcysteine, dithiotreithol (DTT).

20. The method of claim 16, wherein the thiol-containing compound comprises N-acetyl-cysteine.

21. The method of claim 16, wherein the bactericidal compound comprises a hydroxide containing solution.

22. The method of claim 16, wherein the bactericidal compound comprises a sodium hydroxide solution.

23. The method of claim 16, wherein the bactericidal compound comprises a sodium hydroxide and sodium citrate solution.

24. The method of claim 16, wherein the buffer solution comprises a phosphate buffer solution.

25. The method of claim 16, wherein the buffer solution comprises a phosphate buffer solution, wherein the pH of the buffer solution if from about 6.5 to about 7.5.

26. The method of claim 16, wherein the biological sample comprises sputum, bronchoalveolar fluid, gastric lavage fluid, pleural, pericardial or peritoneal fluids or urine.

27. The method of claim 16, wherein one or more of the tubes are adapted for use in a centrifuge.

28. The method of claim 16, further comprising concentrating the mycobacteria.

29. The method of claim 28, wherein concentrating the mycobacteria comprises centrifuging the first container.
